(19) United States
(12) Patent Application Publication
(10) Pub. No.: US 2016/0258869 A1
(43) Pub. Date: Sep. 8, 2016

(54) WATER SOLUBLE PH RESPONSIVE FLUORESCENT NANOPARTICLES

(71) Applicant: University of Utah Research Foundation, Salt Lake City, UT (US)

(72) Inventors: Agnes Ostafin, Salt Lake City, UT (US); Chang-Won Lee, Salt Lake City, UT (US)

(21) Appl. No.: 15/092,271

(22) Filed: Apr. 6, 2016

Related U.S. Application Data
(62) Division of application No. 12/778,798, filed on May 12, 2010.
(60) Provisional application No. 61/326,596, filed on Apr. 21, 2010.
(60) Provisional application No. 61/177,737, filed on May 13, 2009.

(51) Int. Cl. G01N 21/64; G01N 33/84; G01N 33/58; G01N 21/80

(52) U.S. Cl. CPC G01N 21/643; G01N 21/80; G01N 33/84; G01N 33/587; G01N 2021/6439; H01J 37/261

(57) ABSTRACT

A nano-pH sensor can include a nanoparticle having an outer surface functionalized by a carboxy functional group. The nanoparticle is reversibly aggregated as a function of pH and is generally non-toxic. A fluorometer can be oriented to expose the nanoparticles to a light source at a given wavelength. Further, the fluorometer can be configured to detect changes in fluorescence of the gold nanoparticle with changes in pH.

### RELATED APPLICATIONS

This application claims the benefit of U.S. application Ser. No. 12/778,798, filed May 12, 2010 which claims the benefit of U.S. Provisional Patent Application No. 61/177,737, filed May 13, 2009, and U.S. Provisional Patent Application No. 61/326,596, filed Apr. 21, 2010, each of which is incorporated herein by reference in its entirety.

### BACKGROUND OF THE INVENTION

Detection of pH variations at nanoscale resolution poses unique challenges. For instance, in microfluidic devices and biological cells measurement of pH variations could provide insight into function of these devices and the related materials. When the regional dimensions for pH measurement reach the nanoscale, conventional pH detecting methods such as glass electrodes are obviously not usable. Furthermore, the light intensity from small numbers of diffusing pH responsive dye molecules is too low for single or few molecule detection. The signal averaging is further compromised by the tendency of dye molecules to photobleach under prolonged illumination. The process may also release active photoproducts that affect the surrounding pH levels.

The optical properties of gold nanoparticles have been of scientific interest for many years. Most focus has been directed to the properties of their surface plasmon resonance absorbance and Mie scattering which endows gold nanoparticle suspensions with a rich repertoire of colors as a function of particle diameter which respond to solvent conditions such as pH, dielectric function and refractive index. This property has been utilized to generate a variety of pH dependent sensors based on absorbance or Raman scattering techniques. A potential limitation in their use is the lowered sensitivity of detection of light absorbance and light scattering relative to fluorescence in significantly scattering specimens. In general, fluorescence sensing is a more sensitive detection method. However, most gold nanoparticles have no, or only extremely weak photoluminescence. Higher intensity emissions from gold can be seen once quantum confinement effects begin to manifest usually for gold atom clusters between 1.3 nm and 3.0 nm in size. Another way to obtain fluorescence emission is by binding to the particle surface organic molecules which normally have low intrinsic emissions due to intermolecular quenching, and relying on the metal enhancement effect to reduce electron transfer to the chromophore macrocycle.

Most of the types of fluorescing gold species mentioned are prepared and are stable in non-aqueous solutions, limiting their use in biology. To date, the only water soluble gold species with significant emission are those consisting of several, few atom clusters grown in the hydrophobic interiors of water soluble dendrimers and other proteins.

### BRIEF DESCRIPTION OF THE DRAWINGS

The present invention will become more fully apparent from the following description and appended claims, taken in conjunction with the accompanying drawings. Understanding that these drawings merely depict exemplary embodiments of the present invention and they are, therefore, not to be considered limiting of its scope. It will be readily appreciated that the components of the present invention, as generally described and illustrated in the figures herein, could be arranged, sized, and designed in a wide variety of different configurations. Nonetheless, the invention will be described and explained with additional specificity and detail through the use of the accompanying drawings in which:

FIG. 1. Transmission electron microscope image of fluorescent gold nanoparticles.

FIG. 2A is a fluorescence spectrum of gold nanoparticles excited at 280 nm.

FIG. 2B is a fluorescence intensity as a function of sodium borohydrate concentration.

FIG. 2C is an absorption spectra of gold nanoparticles as a function of sodium borohydrate concentration.

FIG. 2D is an excitation spectra of gold nanoparticles.

FIG. 3. Intensity vs. mercaptooctanoic acid to gold ratio; The ratio of MOA to gold is an important factor for the fluorescent gold nanoparticle synthesis. It showed the maximum intensity at 3:1 mercaptooctanoic acid to gold ratio. All the points were measured in triplicate.

FIG. 4. Temperature dependency of the fluorescence emission intensity; The sample was placed in the temperature-controlled antifreeze bath and the fluorescence of the sample was read at each temperature point after being transferred to the fluorometer placed next to the antifreeze bath. Additional 5 min were allowed for the sample cuvette to reach the bath temperature at each temperature point.

FIG. 5. Possible hydrogen bonding between the gold nanoparticles; Carboxylic acid groups on the surface of the gold nanoparticles are accountable for the hydrogen bonding via water molecules between the particles. This hydrogen bonding formation is believed to be the reason for larger diameter size measured from dynamic scattering measurement technique comparing to transmission electron microscopy.

FIG. 6. Size of the gold nanoparticle measured by DLS; It shows infinite increase of the cluster size at lower pH region where charge-charge repulsion is absent. Gold nanoparticle with 3:1 mercaptooctanoic acid to gold ratio was used for the test.

FIG. 7. Photobleaching of gold nanoparticle (solid squares) relative to fluorescein solution (open diamonds); Photobleaching experiment for gold nanoparticles (200 nM) and fluorecein solutions (19 nM) in a 1 cm path length quartz cuvette mounted in a water-cooled jacket at 30° C. The output from a 300W Xenon arc lamp (14200 LUX at the sample position) was directed at the cuvette and the fluorescence spectrum of the sample was measured every 5 minutes. The experiment was performed in water and pH 8.

FIG. 8A is an emission of fluorescence gold nanoparticles as a function of pH.

FIG. 8B shows reversibility of fluorescence intensity as a function of pH. pH was changed from 8 to 5.5 for each cycle.

FIG. 9 shows the zeta potential analysis of the gold nanoparticle by pH change. The zeta potential started to increase from −35 mV at pH 8 and reached close to zero mV at pH 4.

FIG. 10 is a graph of fluorescence versus nanoparticle concentration.

FIG. 11 is a graph of cell viability as a function of nanoparticle concentration.

FIG. 12A is a TEM micrograph of the luminescent gold product produced using reductive aqueous synthesis using a 3:1 molar ratio of gold to ligand. The average particle size is 2.2±0.6 nm.

FIG. 12B is a high resolution TEM micrograph of the particles obtained in FIG. 12A.

FIG. 12C is an ATR-FT-IR spectra of product (solid line) and mercaptooctanoic acid (dotted line). Loss of SH stretch is consistent with the formation of gold-thiol bonding, and shift of C═O stretch and C—H₂ bend vibrations to lower wavenumber with a strong interaction of this molecule with another species, i.e. the gold nanoparticles present in the sample.

FIG. 13A is an excitation spectrum obtained by monitoring the emission at 2.2 eV (˜610 nm) of luminescent gold of the invention. Mark points landmark the known positions of presistent line in Au(0) and Au(I) spectra.

FIG. 13B are electronic energy levels of Au(0) and Au(I) relative to the ligand-metal-metal charge transfer complex (LMMCT) formed by reaction of gold with mercaptooctanoic acid.

FIG. 14A is an excitation spectra obtained for luminescent gold product produced using mercaptododecanoic acid (dotted line), mercaptooctanoic acid (solid line) and mercaptohexanoic acid (dashed line).

FIG. 14B is an emission spectra obtained for the same samples as FIG. 14A. Data is normalized to highlight shape and position changes. In the excitation spectrum MDA is multiplied by 11, and MOA by 3 relative to MHA. In the emission MHA is multiplied by 4 and MDA by 11. MOA is unchanged. This means MOA is more efficiently being sensitized.

FIG. 15A is a schematic of one configuration of gold-mercaptododecanoic acid complexes at the surface of gold nanoparticles.

FIG. 15B is a schematic of one configuration of gold-mercaptohexanoic acid complexes at the surface of gold nanoparticles. The longer chain ligand has greater potential to protect the gold-sulfur bond portion of the molecule from interaction with the surrounding solvent.

FIG. 15C is a schematic of another configuration of gold-mercaptohexanoic acid complexes at the surface of gold nanoparticles.

FIG. 16A is a graph of diameter of particles as a function molar ratio of MOA to gold. Error bars estimated from 3 samples.

FIG. 16B is a graph of relative quantum yield of emission (%) at 610 nm as a function of particle size, Error bars estimated from 3 samples.

### DETAILED DESCRIPTION OF EXEMPLARY EMBODIMENTS

The following detailed description of exemplary embodiments of the invention makes reference to the accompanying drawings, which form a part hereof and in which are shown, by way of illustration, exemplary embodiments in which the invention may be practiced. While these exemplary embodiments are described in sufficient detail to enable those skilled in the art to practice the invention, it should be understood that other embodiments may be realized and that various changes to the invention may be made without departing from the spirit and scope of the present invention. Thus, the following more detailed description of the embodiments of the present invention is not intended to limit the scope of the invention, as claimed, but is presented for purposes of illustration only and not limitation to describe the features and characteristics of the present invention, to set forth the best mode of operation of the invention, and to sufficiently enable one skilled in the art to practice the invention. Accordingly, the scope of the present invention is to be defined solely by the appended claims.

The following detailed description and exemplary embodiments of the invention will be best understood by reference to the accompanying drawings, wherein the elements and features of the invention are designated by numerals throughout.

### DEFINITIONS

In describing and claiming the present invention, the following terminology will be used.

The singular forms “a,” “an,” and “the” include plural referents unless the context clearly dictates otherwise. Thus, for example, reference to “a nanoparticle” includes reference to one or more of such materials and reference to “providing” refers to one or more such steps.

As used herein with respect to an identified property or circumstance, “substantially” refers to a degree of deviation that is sufficiently small so as to not measurably detract from the identified property or circumstance. The exact degree of deviation allowable may in some cases depend on the specific context.

As used herein, “adjacent” refers to the proximity of two structures or elements. Particularly, elements that are identified as being “adjacent” may be either abutting or connected. Such elements may also be near or close to each other without necessarily contacting each other. The exact degree of proximity may in some cases depend on the specific context.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a numerical range of about 1 to about 4.5 should be interpreted to include not only the explicitly recited limits of 1 to about 4.5, but also to include individual numerals such as 2, 3, 4, and sub-ranges such as 1 to 3, 2 to 4, etc. The same principle applies to ranges reciting only one numerical value, such as “less than about 4.5,” which should be interpreted to include all of the above-recited values and ranges. Further, such an interpretation should apply regardless of the breadth of the range or the characteristic being described.

Any steps recited in any method or process claims may be executed in any order and are not limited to the order presented in the claims. Means-plus-function or step-plus-function limitations will only be employed where for a specific claim limitation all of the following conditions are present in that limitation: a) “means for” or “step for” is expressly recited; and b) a corresponding function is expressly recited. The structure, material or acts that support the means-plus function are expressly recited in the description herein. Accordingly, the scope of the invention should be determined solely by the appended claims and their legal equivalents, rather than by the descriptions and examples given herein.

Nano-pH Sensors

A nano-pH sensor can include a nanoparticle having an outer surface functionalized by a carboxy functional group. The nanoparticle is reversibly aggregated as a function of pH. Such nanoparticles tend to be non-toxic, especially for gold nanoparticles. A fluorometer can be oriented to expose the nanoparticles to a light source at a given wavelength. Further, the fluorometer can be configured to detect changes in fluorescence of the nanoparticle with changes in pH.

The nanoparticles can be formed of a variety of materials, as long as the nanoparticles exhibit the desired fluorescence. Non-limiting examples of suitable nanoparticle materials include noble metals such as gold, silver, platinum, iridium, palladium, rhodium, ruthenium and osmium, semiconductors such as CdS, CdSe, ZrO₂ and TiO₂, alloys thereof, intermetallics thereof, and combinations thereof. In one specific aspect, the nanoparticles are comprised of gold. In one aspect, the nanoparticles can be homogeneous, single phase particles which consist essentially of a single material.

Further, the nanoparticles can typically be on the minute end of the nanoparticle size range, although larger nanoparticles may also be suitable for some applications. In one aspect, the nanoparticle has an average particle diameter from about 1 nm to about 10 nm. In another aspect, the average particle diameter is from about 2 nm to about 5 nm.

In one aspect, the outer surface of the nanoparticle can be substantially covered by a plurality of carboxy functional groups. The degree of coverage can vary depending on the conditions used during formation of the nanoparticles. Although other carboxy functional groups can be suitable, mercaptooctanoic acid is particularly suitable. Other non-limiting examples of carboxy functional groups include mercaptoheptanoic acid, mercaptononanoic acid, and the like.

These functionalized nanoparticles can have particularly desirable properties such as strong pH dependent fluorescence, photobleaching resistance, non-toxicity, and the like. Although other emission wavelengths can be used and are typically emitted, the gold nanoparticles generally have a peak emission intensity at about 610 nm. In one aspect, the gold nanoparticle has a photobleaching resistance of 10-15% in 2 hours of illumination using a 300W xenon arc lamp.

The fluorometer can be any fluorometer which is capable of producing an excitation light at a given frequency and detecting fluorescence emission variations. Such devices can range from expensive multi-frequency devices to single frequency devices. A fluorescence intensity sensor can detect changes in fluorescence intensity. By knowing the pH where the minimum and maximum emission is observed it is possible to assign intensities in between these two ranges to a specific pH. The dependence of intensity on pH is sigmoidal over this range. Thus the actual intensity can be calibrated with a two point calibration, and then correlated from a reference curve.

A method of detecting pH can include exposing a plurality of the nanoparticles to a fluid environment. The plurality of nanoparticles can be subjected to an excitation light source having a wavelength. The light source can be visible, infrared, or any other wavelength of electromagnetic radiation which produced a measurable emission from the gold nanoparticles. In one specific aspect, the excitation wavelength is 280 nm. A resulting fluorescence intensity of the excited plurality of nanoparticles can be measured. The fluorescence intensity can be correlated with a pH and visually displayed or otherwise utilized.

The pH measurements can be made in a wide variety of environments. Typically the environments are aqueous although non-aqueous environments can also be suitable. In one aspect, the fluid environment is a physiological environment such as, but not limited to, ex vivo physiological samples, in vivo, or the like. For in vivo applications, the gold nanoparticles can be selectively directed to a particular organ, tissue or area for targeted pH measurement. In one alternative, the nanoparticles can be functionalized with a targeting ligand which selectively binds with a particular tissue. Although other approaches can be suitable, such functionalization can include well established conjugation chemistries using commercial reagents between the exposed carboxylic acid group and the other molecule. Typically, an amide bond is simple to form and most proteins have available amine group for this purpose. A typical exemplary combination involves EDC (1-Ethyl-3-[3-dimethylaminopropyl]carbodiimide hydrochloride)/SHNS (N-hydroxysulfosuccinimide) as a coupling reagents. Other examples can be obtained commercially such as, but not limited to, sulfo-SMCC (Succinimidyl 4-[N-maleimidomethyl]cyclohexane-1-carboxylate), BS³ (Bis[sulfosuccinimidyl] suberate), DSP (Dithiobis (succinimidyl propionate)), DTSSP (3,3″-Dithiobis[sulfosuccinimidylpropionate]), and sulfo-LC-SPDP (Sulfosuccinimidyl 6-(3′-[2-pyridyldithio]-propionamido)hexanoate) which are all commercially available from Pierce Protein Research Products. Where a targeting molecule is designed to be far from the surface, a suitable spacer can be used such as the amino PEG mentioned below. In this case, the amine of the amino PEG can be conjugated to the exposed carboxylic acid group, and the other end of the PEG can be chosen as a more convenient functional group. For example, if an SH group is available, then attachment of a targeting molecule using disulfide bonding is suitable. In another aspect, an avidin-labeled PEG can be constructed so that avidin-biotin recognitions using a biotynylated protein as a targetting molecule can be used. There are numerous alternative conjugation reagents to conjugate various combinations of SH, NH₂, and COOH functional groups. The choice of such coupling reagents can be dictated by the ease and efficiency of the chemistry, and whether the needed reaction conditions would damage either the nanoparticle or the targeting molecule.

The functionalized nanoparticles can be formed in any suitable manner. In one aspect, the nanoparticle can be formed by nucleation, sol gel, attrition or the like. Typically, the formation of the nanoparticles can be substantially simultaneous with introduction of the carboxy functional groups. In one aspect, a solution of the nanoparticle source material can be mixed with a solution of the carboxy functional group source. A reductant can be added with stirring such that nucleation of the nanoparticle and simultaneous attachment of the carboxy functional group ligands occurs. Under reductive conditions, nanoparticle nucleation as well as particle growth takes place alongside ligand charge transfer complex formation. This process can follow a two stage process involving initial formation of noble metal-thiol complexes, followed by slower appearance of nanoparticles accompanied by large increases in photoluminescence intensity. The final product has substantially no free complexes.

In one aspect, folate can be modified with monoamino PEG to introduce alcohol groups on the folate molecule by using EDC and SNHS amide bond coupling agents. Monoamino PEG can then be used to conjugate two carboxylic acid groups from folate and gold nanoparticle. Then these alcohol groups can be used to conjugate to the carboxylic acid groups of the gold nanoparticle surface using ester bond forming coupling agent, DMAP and EDC (Equation I).

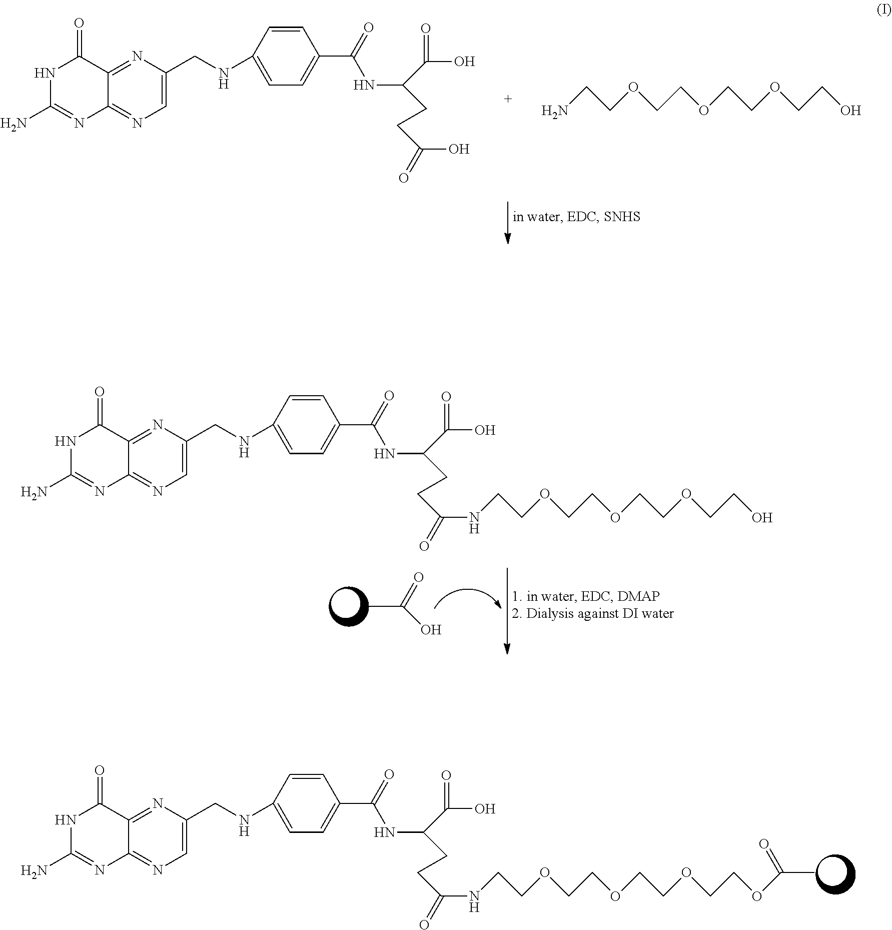

Alternatively, an excessive amount of bis-amino PEG for gold nanoparticle surface coating to cover the surface of the gold nanoparticle with amine groups. After this process, the product will be dialyzed to remove the un-reacted portion of bis-amino PEG and coupling agents such as EDC and SNHS. Then, the surface amine groups of the purified gold nanoparticle will be used for conjugating folate by utilizing its carboxylic acid group (Equation II).

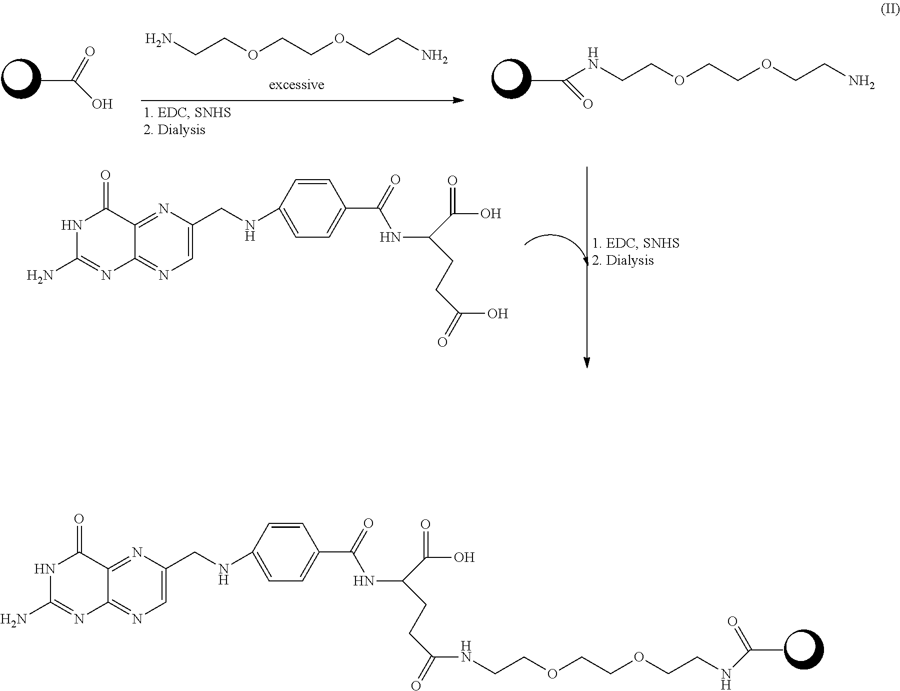

The pH sensor can be formed by providing a plurality of the nanoparticles and providing a fluorometer configured to detect changes in fluorescence of the nanoparticle with changes in pH in a fluid environment. In one example, the plurality of nanoparticles can be formed by simply mixing a gold salt, a mercaptooctanoic acid, and a reducing agent. Similar mixing schemes can be devised for other nanoparticle and/or carboxy functional group materials are chosen. Typically, the reducing agent is present in excess.

In one aspect, the gold salt can be any reducable gold salt. However, in one specific aspect non-limiting examples of suitable gold salts can include tetrachloroaurate hydrate or gold chloride. The mercaptooctanoic acid can typically be 8-mercaptooctanoic acid. Mercaptooctanoic acid has been chosen due to its structural simplicity which just has one thiol and one carboxylic acid group at each end of chemically inert octane backbone. Thus it is unlikely that this structure is capable of nucleating several gold atomic clusters. In one aspect, the reducing agent is sodium borohydride. Other non-limiting examples of suitable reducing agents can include other borohydrides, sodium citrate, sulfate, thiolates, and weak alcohols. Specific examples of such reducing agents can include, but are not limited to, LiBH₄, citric acid, lithium citrate, Na₂SO₄, Li₂SO₄, alkanethiols, ethanol, methanol and mixtures thereof. In some cases the capping agent (e.g. the function performed by mercaptooactanoic acid) can also function as a reducing agent. In this case part of the molecule has to be able to react with the gold ions in a similar fashion.

The ratio of gold to mercaptooctanoic acid can affect fluorescence performance. In one aspect, the ratio of gold to mercaptooctanoic acid can be from about 1.5:1 to about 10:1, with a particular effective range from about 2:1 to about 5:1 and in one aspect about 3:1.

Although the nanoparticles can be used as formed, it is often desirable to further purify the nanoparticles substantially remove impurities which might reduce sensitivity, increase toxicity, or otherwise introduce unpredictable effects on fluorescence and pH sensing performance.

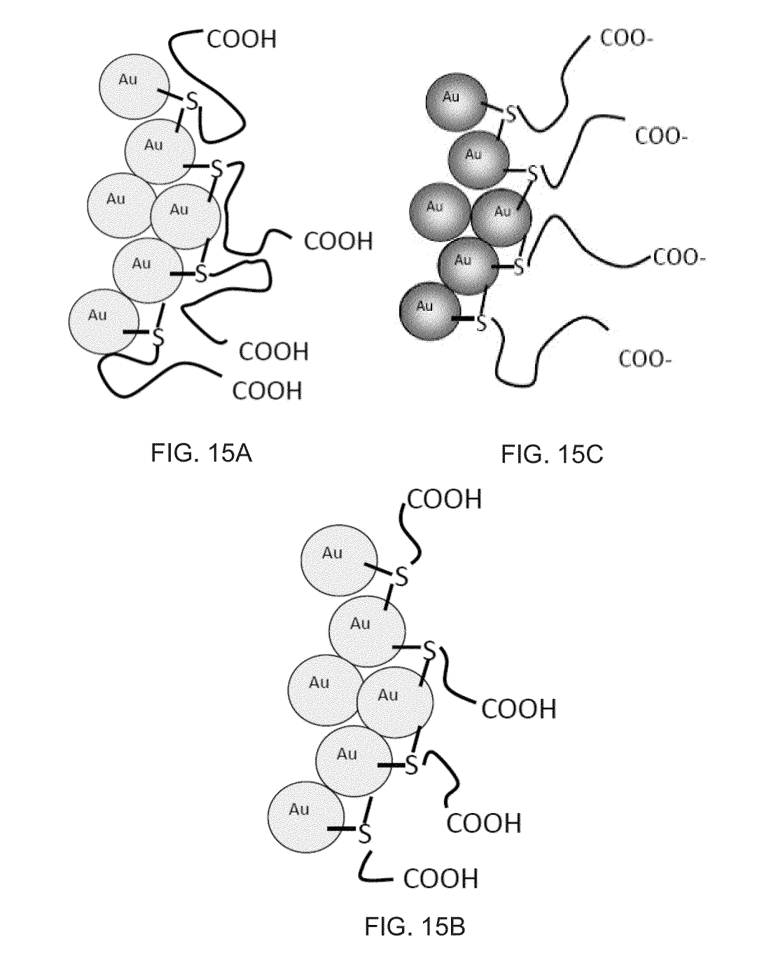

In one aspect, the nanoparticles can be induced to form a molecular brush structure (e.g. FIGS. 15A and 15B). These structures can have differing configurations under varying pH conditions. FIG. 15A illustrates an example of a gold-MOA nanoparticle molecular brush under low pH, while FIG. 15C illustrates the same molecular brush under high pH conditions. In the low pH configuration, the deprotonation of the carboxylic acid group of MOA causes a collapse of the molecules onto the nanoparticle surface. This obscures the emitting part of the molecule from the polar surroundings and induces a polarity-consistent change in position and shape of the excitation spectrum and change in emission intensity. The high pH configuration has the molecules extended such that the emitting portions of the molecule are exposed to surrounding environment. The pH response of this system between pH 5 and 8 was found to be reversible with no aggregation of suspended materials.

The nanoparticles can be used for a variety of applications. The nanoparticles can be present in the fluid environment at various concentrations based on the desired emission intensity, toxicity, and other factors. Typically for physiological fluids, a concentration from about 100 nM to about 500 nM can be suitable. In another aspect, the fluid environment is an industrial environment such as, but not limited to, industrial effluent, mine run-off, product samples, intermediate samples, and the like.

The nano pH sensors and functionalized nanoparticles can be used in a wide variety of environments including identification and monitoring of cancerous cells, tracers in microfluidic devices, biosensors, and the like. For example, solid tumors can have a substantially increased extracellular pH gradient as compared to normal tissue. These nano pH sensors can also be used for imaging with targeted delivery to specific sites, for example, to evaluate the consequences and causes of diseases such as cancer, to study the mechanisms of new drugs, or to follow the events of DNA and protein synthesis.

### Example 1

Mercaptooctanoic acid (C₈H₁₆O₂S), hydrogen tetrachloroaurate (HAuCl₄), sodium borohydrate (NaBH₄) and all solvents were purchased from Sigma (St. Louis, Mo.). E-Pure filtered water (18 MΩ) was used for all syntheses. Dialysis membranes (1000 MWCO) were obtained from SpectraPor (Rancho Dominguez, Calif.) and rinsed in E-Pure water before use. Formvar-coated carbon TEM grids were obtained from Ted Pella (Redding, Calif.).

Gold nanoparticles were synthesized by mixing 2.30 mL of 0.01087 M gold solution (0.025 mmole) in water in a 25 mL Erlenmeyer flask using 1.5 cm stir bar spun at 600 rpm using a magnetic stirrer. To this was added 12.8 μL of mercaptooctanoic acid (0.075 mmole) in 0.45 mL of ethanol). Ten seconds after the mercaptooctanoic acid addition, 1.75 mL of 0.143M sodium borohydride (0.25 mmole) was added and the mixture stirred overnight at room temperature after using Parafilm to seal the top of flask to prevent evaporation. The total reaction volume was 4.5 mL. The next day, sample was separated into three 1.5 mL centrifuge tubes and centrifuged at 12000 rpm for 10 min at 25° C. The yield of fluorescent gold product was determined by subtracting the weight of the dried pellet from the initial amounts of reactants. Typically, the percent yield of final product was 81.1±4.1%. The supernatant was triturated with 200-proof EtOH leaving the product pellet to dry. The pellet was resuspended in E-pure water for the concentration of 0.2 mg/mL.

Transmission electron micrographs were obtained using a JEOL JSM840a TEM at the Electron Microscopy facility at Brigham Young University, Provo, Utah. A drop of 5 μL gold nanoparticle solution was dried on a Formvar TEM grids the grid at room temperature. Particle size distributions were obtained using dynamic light scattering with a Malvern Zetasizer NanoZS (Worcestershire, UK) in 1 cm quartz cuvettes at 20° C. Typical polydispersity of the samples was 0.4 to 0.6. The number of scans to be averaged was determined automatically by the machine depending on the output quality and was usually in the range of 12-17.

There is a difference in TEM size of the gold nanoparticle and the size distribution measured by DLS method. DLS uses laser of 633 nm (red) and there is no absorbance in gold nanoparticle for that wavelength range. So, it is unlikely that the laser interferes with the gold nanoparticle and affecting the results.

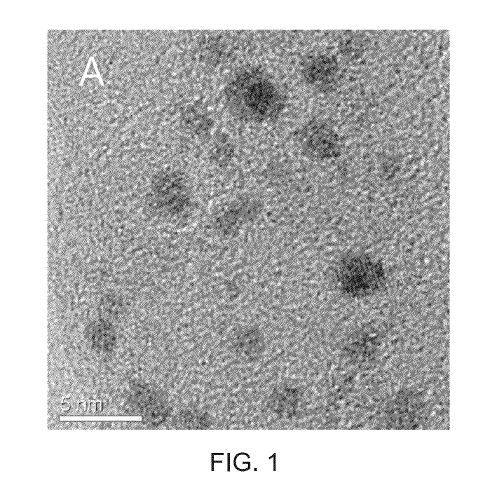

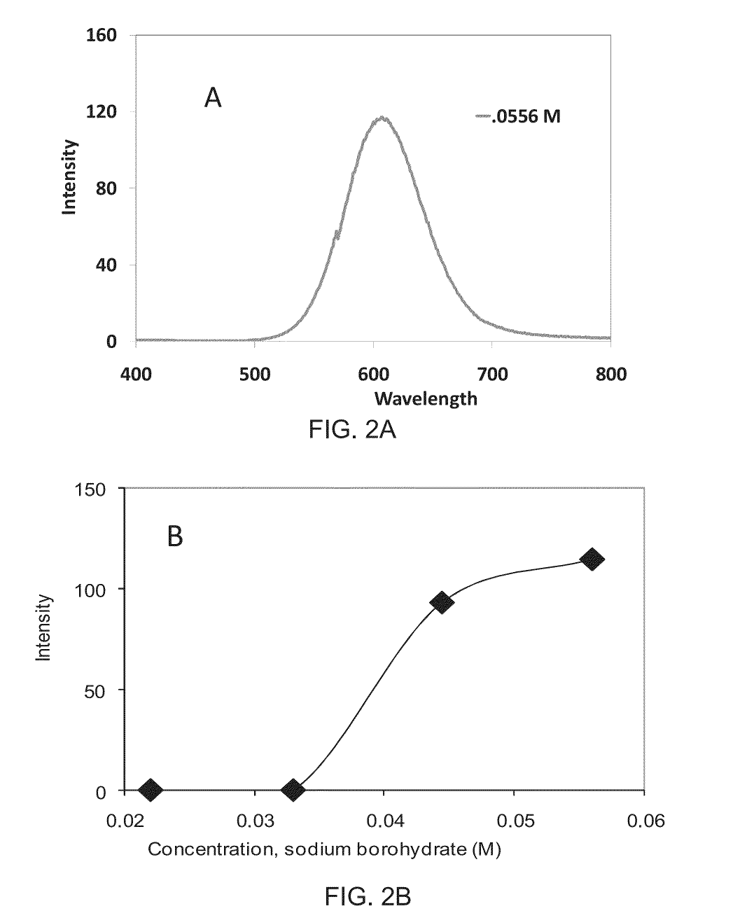

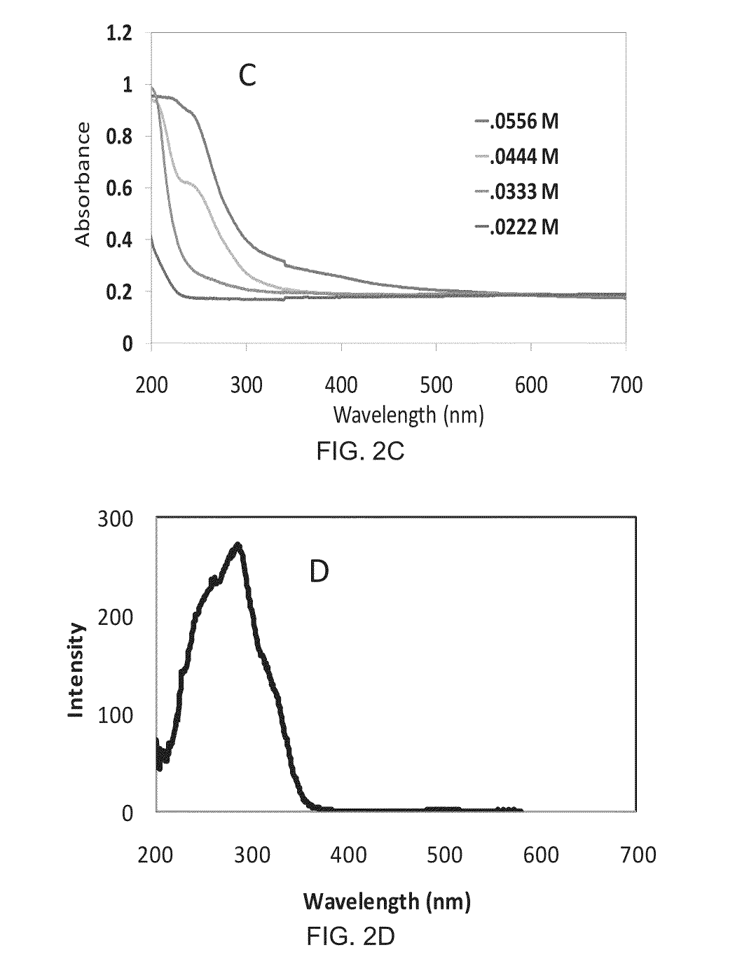

As shown by the result of high resolution transmission electron microscopy (FIG. 1), the fluorescent gold nanoparticles produced using this synthesis procedure, have an intrinsic particle diameter at 2.2±0.6 nm. When placed onto a UV transilluminator with excitation wavelength 254 nm they produce a bright red emission. The spectrum of this emission is shown in FIG. 2A is centered around 614 nm with a band width of about 100 nm. The intensity of this fluorescence emission was found to depend on the amount of sodium borohydrate that was used in the synthesis (FIG. 2B). From the corresponding absorption spectra (FIG. 2C) it is clear that the emission is correlated with the appearance of a new absorbance between 260 nm and 300 nm. The excitation spectra obtained for a red emitting gold sample (FIG. 2D) showed a strong peak in the emission output at around 280 nm.

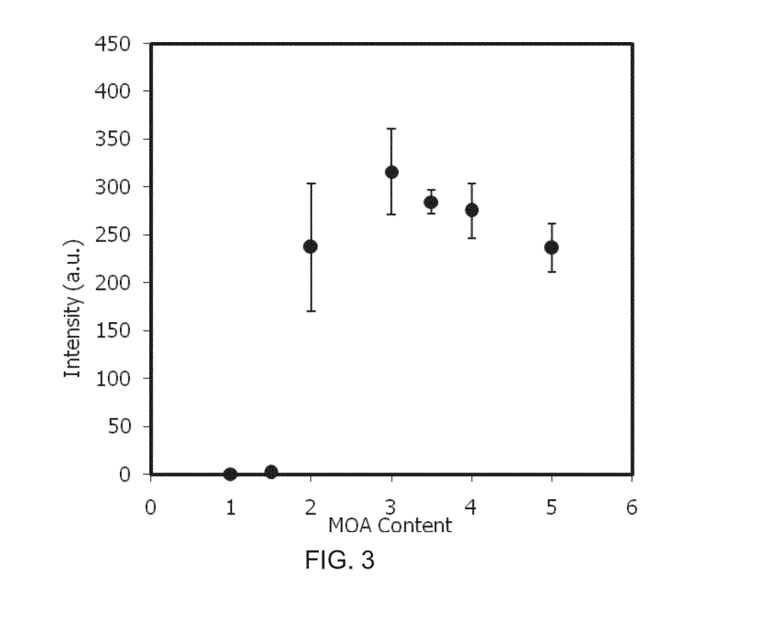

The synthesis of the gold nanoparticle is also dependent on the input ratio of mercaptooctanoic acid to gold (FIG. 3). As illustrated in FIG. 3, gold nanoparticles with X:1 (X=1, 1.5, 2, 3, 3.5, 4 and 5) with X being a ratio of mercaptooctanoic acid to gold were synthesized. Even though an emission peak shift was expected with different ratio of the capping agent, mercaptooctanoic acid to gold, the emission peak did not appear to be strongly shifted. There was only a minimal emission peak shift monitored from 608 nm (2:1) to 613 nm (5:1) (data not shown). However, these results showed difference in terms of emission intensity with maximum peak shown at 3:1 ratio.

Relative quantum yield (QY) of the gold nanoparticle was calculated as 0.01. Fluorescein dye (QY=0.95) was used as a known reference dye molecule. The following equation was used for the calculation.

$$Φ = {Φ}_{R} \frac{Int}{{Int}_{R}} \frac{{A}_{R}}{A} \frac{{n}^{2}}{{n}_{R}^{2}}$$

Here Φ is quantum yield of sample, subscript R is denoting quantum yield of reference molecule, Int is fluorescent intensity, A is absorbance and n is refractive index. This quantum yield was calculated based on the known values of refractive index of gold solution (0.47) and fluorescein solution in water (1.3335). The refractive index value for the gold nanoparticle capped with mercaptooctanoic acid may have different refractive index compared to the gold solution.

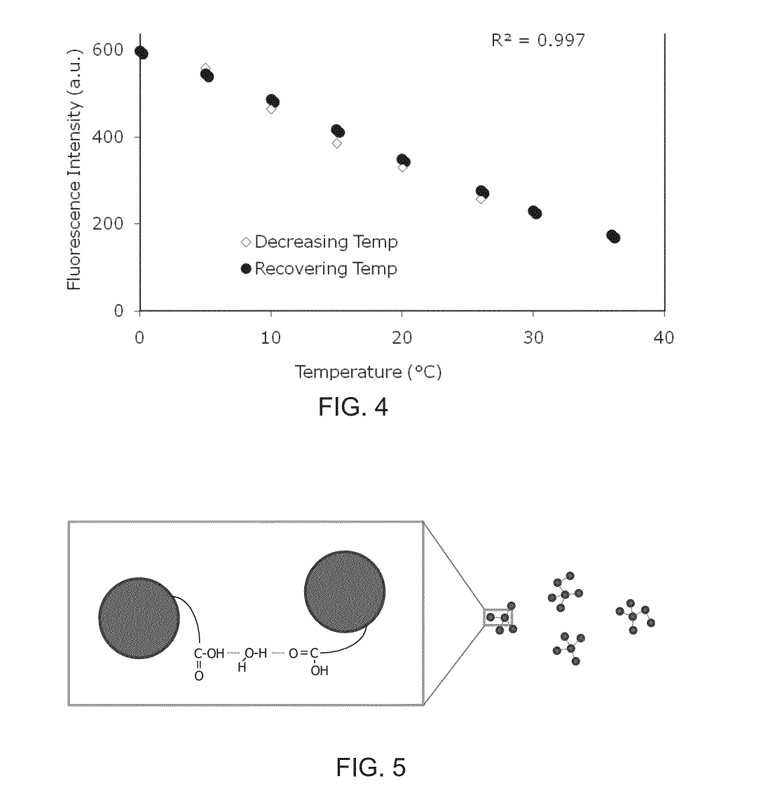

Reversibility dependence on temperature from 0° C. to 36° C. was also performed to understand the relationship between the temperature and the fluorescence intensity (FIG. 4). This relationship is considered to be an indicator of measuring quantum efficiency of the fluorescence nanoparticles.

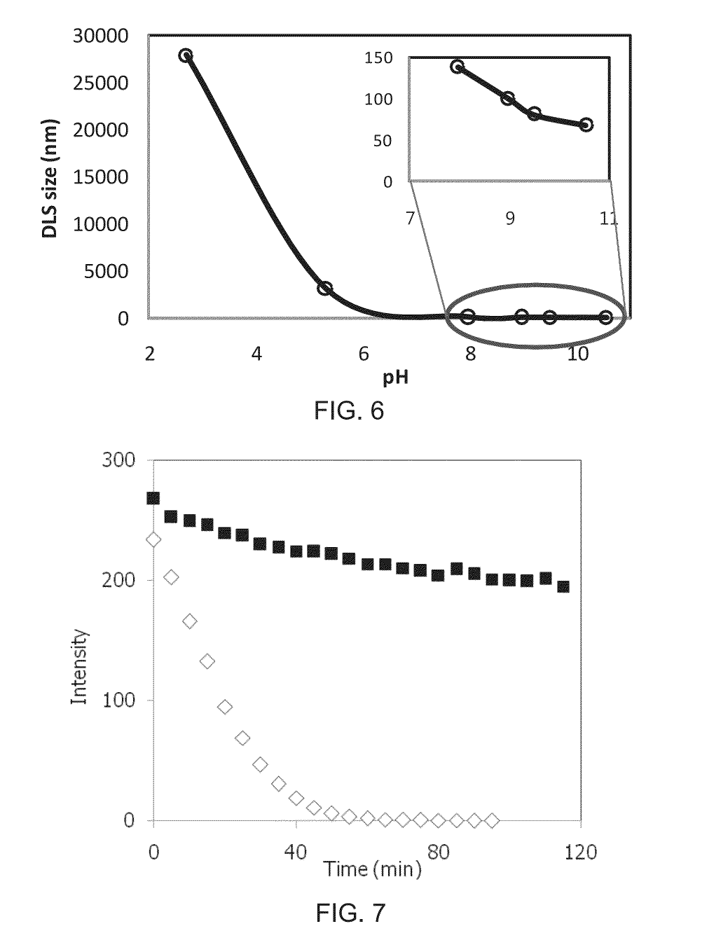

The intrinsic difference of the gold nanoparticle size in solution and dry states may be caused by loosely bound clusters of gold nanoparticle by hydrogen bonding of carboxylic acid groups on the gold nanoparticle surface. Even though there is repulsion in a higher pH range due to negatively charged COO— groups, it may still affect the cluster formation (FIG. 5). Due to the repulsion of the COO— groups, the growth of the cluster is likely to be balanced at certain point, which is the point measured from DLS. In the absence of the charge-charge repulsion at lower pH, a dramatic increase of the cluster size of the gold nanoparticles was measured by DLS (FIG. 6).

Absorption spectra were recorded between 200-800 nm using a Shimadzu UV mini 1240 (Kyoto, Japan) absorption spectrophotometer, from Fisher Scientific, in 1 cm path length quartz cuvettes at 20° C. The spectrometer resolution was +/−2 nm. Fluorescence spectra from 400 nm to 800 nm at 20° C. were obtained using an excitation wavelength of 280 nm, using a Cary Eclipse spectrophotometer from Varian (Palo Alto, Calif.) in 1 cm quartz Suprasil cuvettes. Slit width was fixed to 5 nm and the spectral resolution was 1 nm.

By understanding the temperature dependent fluorescence emission behavior of the gold nanoparticle system, we can estimate its energy transfer efficiency. The effect of the temperature for the quantum yield of the fluorescence (Φ_{f}) is described as the following equation where k_{f} is the rate constant of the fluorescent and k_{d} is the rate constant of deactivation by all competitive non-radiation processes including heat.

$${Φ}_{f} = \frac{{k}_{f}}{{k}_{f} + Σ {k}_{d}}$$

Thus, with increased temperature leads a smaller overall apparent quantum yield. It is known that a decrease in fluorescence emission intensity corresponds with increased measuring temperature with and without the change in peak position. This example shows that there is linear dependency on the temperature and fluorescence intensity and it requires 24.7 K increase for 50% decrease of the fluorescence emission intensity.

Photobleaching tests were performed at constant temperature of 30° C. Specimens were placed in a 1 cm path length Suprasil quartz cuvette and mounted in a water-cooled jacket. The output from a 300W Xenon arc lamp was directed at the cuvette and the fluorescence spectrum of the sample was measured every 5 minutes. The light intensity was 14200 LUX at the point of exposure determined by digital illumination meter DX-200 from Edmund Optics (Barrington, N.J.). Photobleaching measurements (FIG. 7) showed that the gold nanoparticle emission was significantly more stable than compared to fluorecein dye, losing only 20% of its initial emission intensity after 130 minutes of illumination compared to fluorescein dye which lost 100% of its emission after 40 minutes.

The MOA-gold nanoparticle system was compared with organic dye, fluorescein for the photobleaching test and as shown in FIG. 7, the MOA-gold nanoparticle system possesses superior photobleaching property comparing to organic dyes. It is believed that more number of metallic bonding of the core gold nanoparticles as well as covalent bonding between organic molecules and gold surface create more stable fluorophore for the system comparing to a single-molecule fluorophore of organic dyes.

To explain the origin of the fluorescence in the gold nanoparticle system, it is theorized that the carboxylic acid groups of the surface of gold nanoparticles play an important role for the fluorescence behavior. Excited electrons can be generated from the carboxylic groups near the surface of the gold cluster and then transferred to the gold surface to follow the emitting process. The large Stokes shift with distinct clear edge of the excitation spectrum would be the result of the transfer. Unlike typical quantum dots which have continuous excitation spectra without any peak like shape, MOA-gold nanoparticle system has a clear edge around 350 nm and peak around 280 nm region. Surface bound mercaptooctanoic acid group via covalent Au—S bonding appears to be responsible for the excitation process while the gold surface and possibly its defect sites would be responsible for the emission process after the excited electrons being transferred to the gold surface. Further, at higher pH, the carboxylic acid groups will be deprotonated thus the surface of the gold nanoparticle will be negatively charged. The charge-charge repulsion becomes greater than hydrophobic interaction of octanoic acid chain which makes the particles separated and well dispersed. Meanwhile, at lower pH, the carboxylic groups at the surface of the nanoparticle will be protonated and neutralized. With the neutralized charge at the end of the capping molecule, hydrophobic interaction and possible hydrogen bonding interaction dominates which eventually results the aggregation of the particles. This aggregated particle cluster can block and scatter the excitation as well as emission lights to/from the individual nanoparticle so that the emission intensity attenuates.

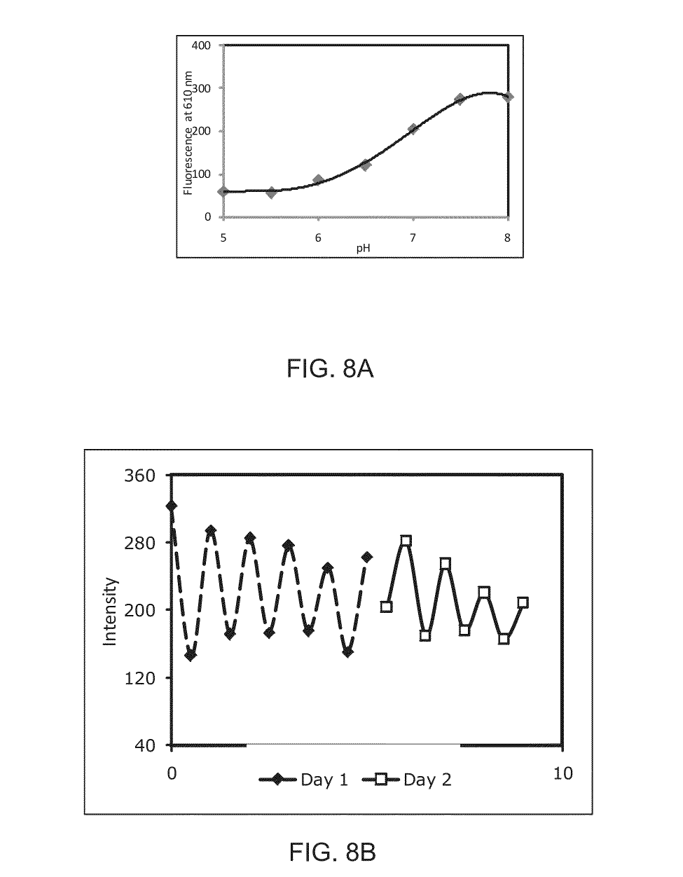

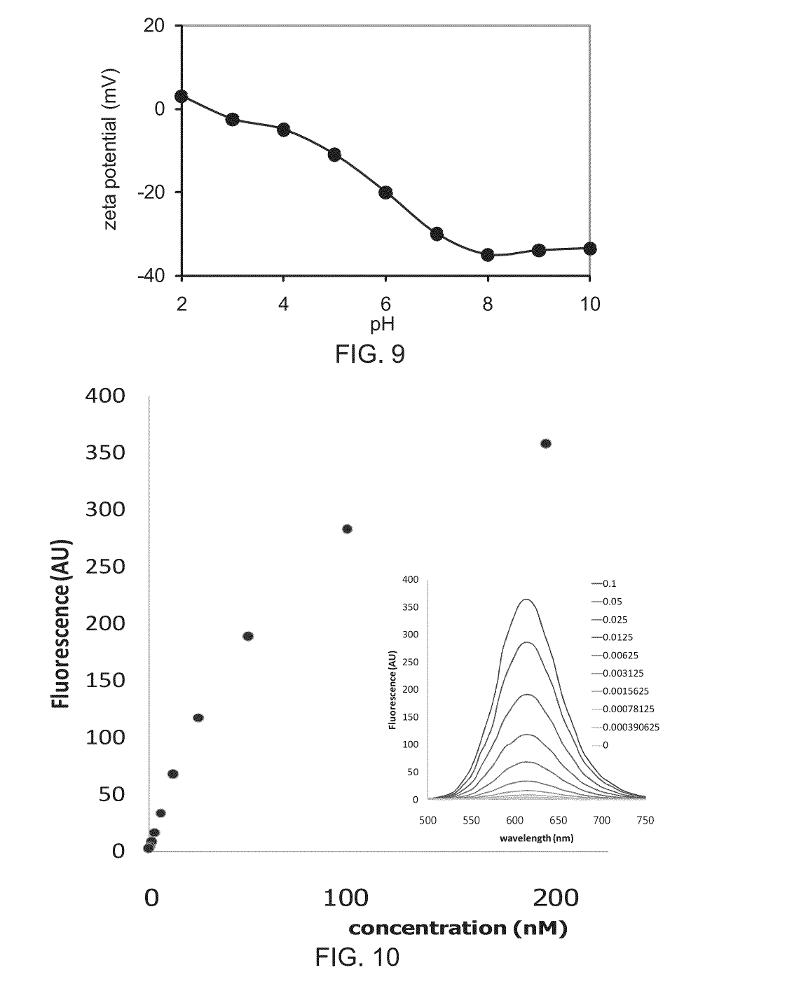

The sample was titrated with 1M NaOH to pH 8 and the fluorescence intensity and absorbance were measured. The sample was then titrated with 1M HCl in steps of pH 0.5 down to pH 5 and back up to pH 8 using 1M NaOH. The fluorescence and absorbance were measured at each point. The reversibility test from pH 8 to pH 5.5 was performed to measure the stability of the particles by changing the pH by titirating with 1M NaOH and HCl. The fluorescence was measured at the highest and lowest point from each cycle. Zeta potential analysis was performed using Malvern Zetasizer NanoZS. Fluorescence as a function of pH was measured and is shown in FIG. 8A. FIG. 8B illustrates intensity as a function of the number of cycles to show reversibility of this nanoparticle system. FIG. 9 illustrates the zeta potential as a function of pH to confirm correlation with intensity. FIG. 10 also illustrates the effect of nanoparticle concentration on emission. The inset graph illustrates the same information as a function of wavelength.

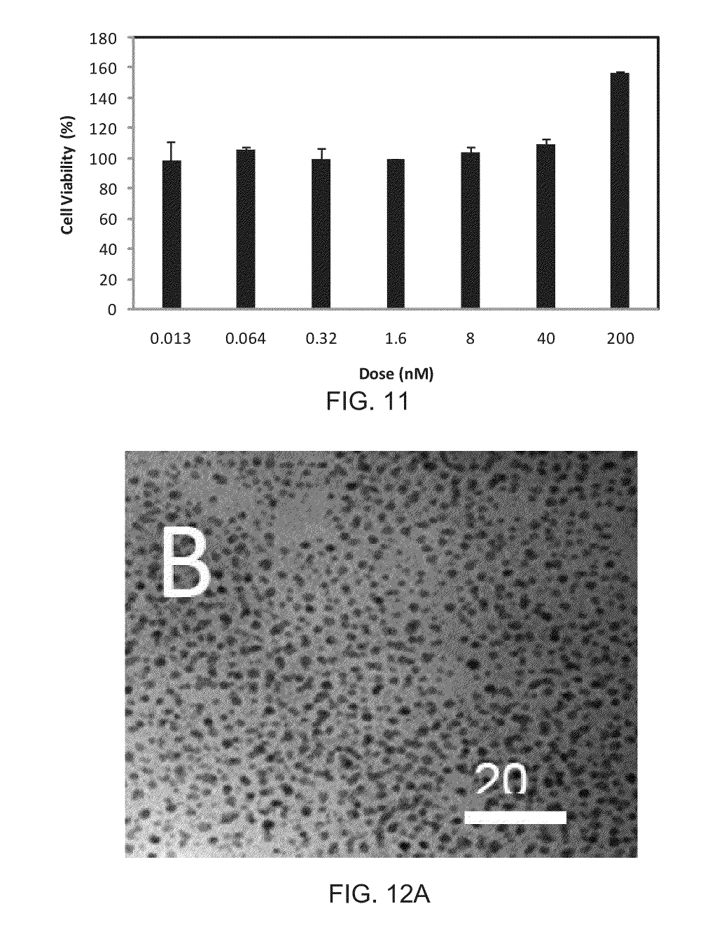

Cell viability measurements were performed using the Dojindo methods to assess toxicity toward BEAS-2B (lung epithelial cells) up to concentrations of 200 nM. The results are shown in FIG. 11. It was noted that simple aggregation does not cause the emission to change, but pH change which may be accompanied by aggregation does. Thus it appears that the protonation state of the COOH group on the mercaptooctanoic acid is a responsible parameter determining the intensity of the emission.

### Example 2

Mercaptooctanoic acid (C₈H₁₆O₂S) (MOA), mercaptohexanoic acid (C₆H₁₂O₂S) (MHA), mercaptododecanoic acid (C₁₂H₂₄O₂S) (MDA), mercaptopropanoic acid (C₃H₆O₂S) (MPA), hydrogen tetrachloroaurate (HAuCl₄), sodium borohydrate (NaBH₄), ethanol (EtOH), fluorescein and all solvents used in this example were purchased from Sigma (St. Louis, Mo.). Precast Tris-HCl 4-15% gradient gel, tris-glycine SDS buffer and Laemmli sample buffer were purchased from Bio-Rad (Hercules, Calif.). Formvar-coated carbon TEM grids were obtained from Ted Pella (Redding, Calif.).

The synthesis for gold nanoparticles involved mixing 2.30 mL of 0.01087 M HAuCl₄.3H₂O solution (9.8 mg, 0.025 mmole), with 1.75 mL of water solution containing various amounts of sodium borohydride (0.375 mmole, 0.25 mmole, 0.2 mmole, 0.15 mmole, 0.1 mmole), and 0.45 mL of ethanol solution containing various amounts of the corresponding mercaptoalkane carboxylic acid (0.025 mmole, 0.0375 mmole, 0.05 mmole, 0.075 mmole, 0.0875 mmole, 0.1 mmole, 0.125 mmole). For some syntheses, 0.45 mL of ethanol solution containing mercaptopropanoic acid, mercaptoheptanoic acid, or mercaptododecanoic acid (0.025 mmole) was added instead of mercaptooctanoic acid. Reactions were performed in low intensity, indirect light to reduce photodecomposition effects, and specimens were stored in the dark.

The sequence of mixing was the addition of mercaptoalkane carboxylic acid solution to a previously prepared gold solution, followed by addition of NaBH₄ solution, each added within 10 seconds of the other. Most experiments were performed using mercaptooctanoic acid unless otherwise indicated. Parafilm® was used to seal the top of flask to prevent evaporation while the mixture was stirred overnight at 600 rpm at 25° C. The next day, the sample was separated into three pre-weighed 1.5 mL centrifuge tubes and centrifuged at 12,000 rpm for 10 min at 25° C. to pellet any large particulates that might have formed. The supernatant containing the fluorescent product was decanted and triturated twice using 200-proof EtOH, allowing the fluorescent product to precipitate and be pelleted by centrifugation (12000 rpm, 10 min) using an IEC Micromax RF bench-top centrifuge (Thermo Scientific, Waltham, Mass.). This purified pellet was dried under vacuum for 3 days and was resuspended in E-pure water to make a concentration of 0.2 mg/mL. The final pH of the gold nanoparticle suspension was 8.5 and all the experiments were conducted at this pH unless noted otherwise.

SDS-Page gel electrophoresis was performed using a Mini-Protean 3 cell from Bio-Rad (Hercules, Calif.) using precast Tris-HCl 4-15% gradient gel, Tris glycine SDS buffer and Laemmli sample buffer. To prepare the sample mixture, a triturated pellet of mercaptooctanoic acid stabilized gold nanoparticles containing 1 μM particles was redispersed in 1 mL in E-pure water and mixed with sample buffer at 1:1 (v/v) ratio. Then, 30 μL of sample mixture was loaded in the well of the gradient gel and 200V applied for 1 hour until the smallest size marker reached the bottom of the well. The gel was carefully removed from its cassette and luminescence from the gold nanoparticles observed by placing the gel on the surface of a UV transilluminator (White/UV transilluminator, Upland, Calif.) providing 254 nm excitation light. The luminescent image was recorded using a Canon digital camera for further analyses. The size ladder in the gel could be observed via absorbance of the UV light as dark lines seen in the image.

ICP-MS analysis using an Agilent 7500ce mass spectrometer (Santa Clara, Calif.) was used to obtain the gold (m/z 197) content of aqua regia-digested samples. The samples were diluted (1 in 100) in 5% HNO₃ and run together with a calibration curve prepared from a soluble gold standard (Inorganic Ventures, Madrid, Spain). Iridium (3.3% in HCl, Inorganic Ventures) was used as internal standard (m/z 192). A self-aspirating PTFE nebulizer (ESI Scientific), PTFE cyclonic spray chamber (PC3 Elemental Scientific), and platinum cones were used. For sulfur (m/z 32) content analyses, the samples were diluted (1 in 2) using 2.4% HNO₃ and run together with a calibration curve prepared from a soluble sulfur standard (H₂SO₄, Inorganic Ventures). In this case terbium (m/z 159) was used as internal standard. To discount any interference of gold in the solution on the detection of sulfur, a known amount of sulfur standard (10 ppm) was mixed with the different concentrations of gold (0, 5, 10, 15 and 20 ppm). Results showed there was no interference effect.

Transmission electron microscopy images were obtained using a JEOL JSM840a TEM at the Electron Microscopy facility at Brigham Young University, Provo, Utah and Tecnai T12 TEM (Philips, Andover, Mass.) at the Heath Science Core Facility in the University of Utah. A 5 μL drop of gold nanoparticle solution was allowed to air-dry on the center of the TEM grid at room temperature.

FT-IR spectra were obtained using a Varian 660-IR measured by putting a drop of concentrated mercaptooctanoic acid stabilized gold nanoparticle in water (2 mg/50 μL) on the crystal of an ATR cell (Varian 3100 FT-IR, Palo Alto, Calif.). Pure mercaptooctanoic acid and water were also analyzed as controls.

Absorption spectra of the gold nanoparticle suspensions containing a calculated 1 μM of particles at pH 8.5 and pH 3 were recorded between 200-800 nm at room temperature, using a Shimadzu UV mini 1240 (Kyoto, Japan) absorption spectrophotometer, in 1 cm path length quartz cuvettes at 20° C. Spectrometer resolution was ±2 nm.

Fluorescence emission spectra of gold nanoparticle suspensions containing a calculated 1 μM of gold particles were obtained from 400 nm to 800 nm at 20° C. using a Cary Eclipse spectrophotometer from Varian (Palo Alto, Calif.). An excitation wavelength of 290 nm was used. Samples were placed in a 1 cm quartz Suprasil cuvettes, and spectrometer slit width was fixed to 5 nm to obtain a spectral resolution of 1 nm. The excitation spectra monitoring 610 nm emission as a function of excitation wavelength between 200 and 600 nm were taken using the same instrument. All spectra were obtained at pH 8.5 unless otherwise noted.

The relative emission quantum yield of the gold nanoparticle stabilized with mercaptooctanoic acid was calculated in reference to the known quantum yield, 0.95, of an aqueous fluorescein dye solution. A series of gold suspensions were prepared by diluting the as-prepared gold suspension by 2 to 20 times and the fluorescence yield relative to absorbance was measured. The maximum fluorescence yield was obtained by extrapolating the curve to infinite dilution in order to avoid problems with self-quenching. A similar plot and extrapolation was obtained for fluorescein solutions prepared over the range of 0.01 to 1 uM in concentration. The relative quantum yield of the gold product was obtained as in Example 1.

Gold nanoparticle suspensions were placed a UV-transparent cuvette and placed 2 cm in front of a photomultiplier tube (R636P, Hamamatsu Photonics, Hamamatsu, Japan) which had a 10 ns rise time. The excitation wavelength at 266 nm was generated as the fourth harmonic of a Nd:YAG regenerative amplifier (4400 series, Quantronix, East Satauket, N.Y.). The average laser power was measured to be 0.2 mW. A repetition rate of 770 Hz was used. The photomultiplier signal was collected with an SR400 photon counter (Stanford Research Systems, Inc., Sunnyvale, Calif.) triggered by the excitation pulse using a moving gate. Exponential decay fitting of the following equation,

y=A*exp(−x/t)+B

was used to obtain the lifetime of the emission from the recorded data.

### Discussion of Results

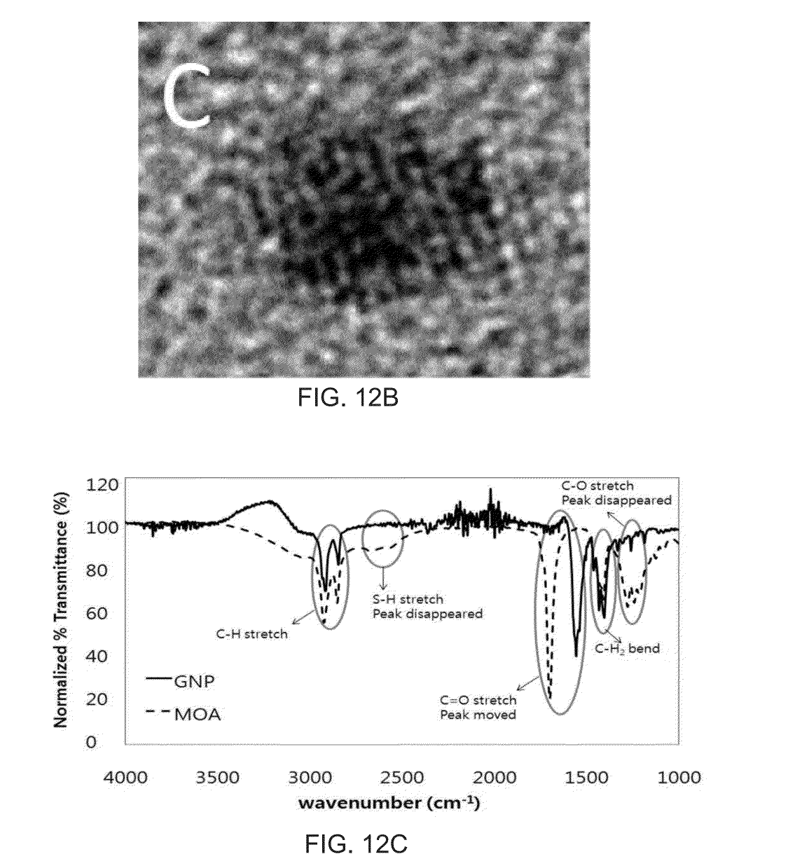

The final photoluminescent product produced after 48 hours of reaction, and purified with trituration, exhibited an absorbance maximum at 290 nm and emission at 610 nm. Electrophoresis showed that the material consisted of substantially monodisperse nanoparticles with electrophoretic migration properties similar to that of MW markers ˜120 kDa in size. For an Au33 cluster, the reported electrophoresis band position was 10 kDa, so the 120 kDa band observed here corresponds to a particle with over 300 Au atoms. This value is also consistent with the particle size estimated from TEM analyses (FIG. 12A). Notable, was the absence of any bands in the 10-30 kDa range which are typical of molecular-sized ligand complexes.

HRTEM showed particles with a clearly visible atomic lattice (FIG. 12B). Detailed analysis of the images using Photoshop (Adobe, San Jose, Calif.) allowed the estimation of the average d-spacing of the gold lattice in the nanoparticles of ˜0.22-0.24 nm. This value is very close to that of other reported Au (111) lattice spacings (0.235 nm and 0.312 nm (26)). Using the density of pure gold, 19.32 g/cm³, one Au atom should occupy 0.017 nm³. Since the volume of a 2.2 nm diameter (radius, r=1.1 nm) gold nanoparticle is 5.58 nm³, the number of Au atoms in a 2.2 nm diameter gold nanoparticle should be about 333. Therefore, both TEM and electrophoresis measurement were in agreement. ICP-MS analyses of gold and sulfur content in the final product showed that the gold to sulfur molar ratio was 1:0.27.

ATR-FTIR spectrum of the purified photoluminescent product was compared to that of pure mercaptooctanoic acid (FIG. 12C). The result indicated the loss of SH stretch vibrations in the product sample, which is consistent with the formation of gold thiol ligand complexes. A shift of the characteristic C═O stretch and CH₂ bend vibrations to lower wave number are consistent with a strong interaction of this molecule with another entity, which appears to be an underlying gold nanoparticle support.

MOA has no appreciable emission of its own, and its HOMO-LUMO gap energy obtained experimentally and by calculations is in the far UV. Simulated orbital isosurfaces obtained using the Gaussian 3 program show that for unbound MOA, the LUMO orbital is located at the carboxylic acid group and the HOMO orbital is located at the thiol end region of the molecule. When complexed to one or more gold atoms, a significant change in orbital distribution takes place, and the isosurface plot shows that both molecular orbitals become located at the sulfur end of the molecule. This difference results in a shift of the HOMO-LUMO energy gap to lower energy.

The spectral trends of unbound MOA and gold-complexed MOA produced in this example in solvents of different polarity are also consistent with a major difference in orbital distribution of the two species. As solvent polarity increases, the energy of the singlet excited state of MOA shifts to higher energy. A blue shift of the excitation spectrum with increasing polarity is rather unusual and indicates that the absorbing species is already quite polar and additional stabilization by dipole-dipole interactions with water has minimal effect.

The final product produced in this example exhibited an excitation spectrum with maximum at ˜4.3 eV (290 nm), and an emission maximum at 2.03 eV (610 nm). Complexes with an intrinsic absorbance maxima at around ˜3.1 eV (400 nm), and very weak emission at ˜2.7 eV (477 nm) are of the type Au—SR and are referred to as LMCT complexes, while those with an absorbance at ˜3.9 eV (320 nm) and a very intense emission ˜1.94 eV (640 nm) are LMMCT with configurations. Variations in these parameters are sometimes attributed to mixing with metal centered orbitals. Based on the emission wavelength and emission lifetime of 1.45 μs this appears to be an LMMCT species.

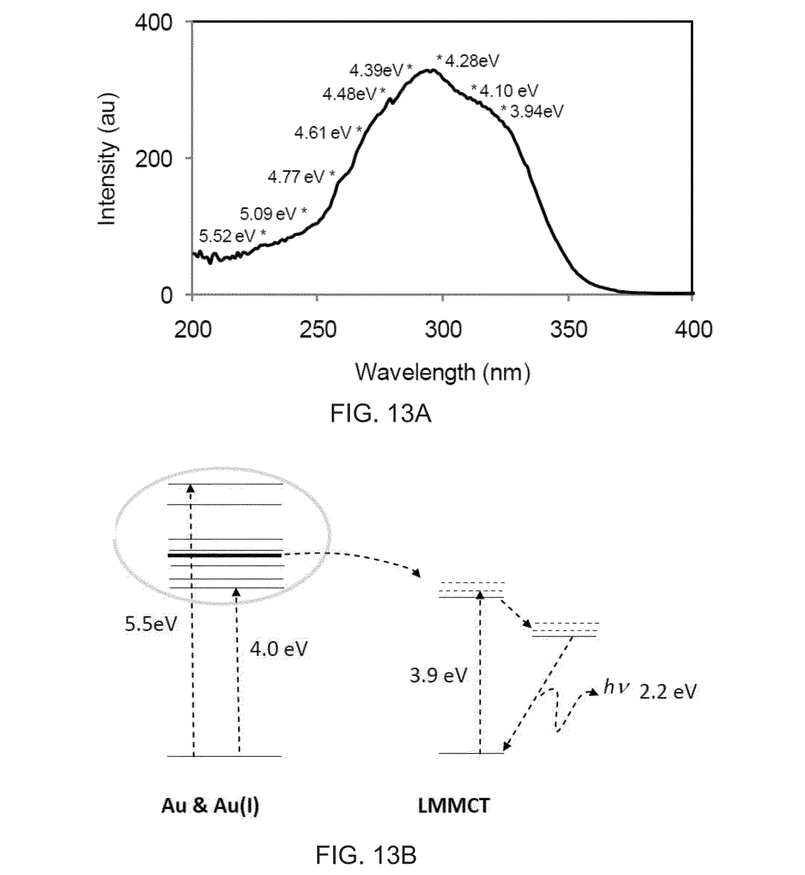

The blue-shifted excitation energy maximum to 4.3 eV (290 nm) for the complexes produced here compared to that of other reported ligand complexes can be understood in terms of overlap of the electronic orbitals of gold atoms and CT complexes with the resulting wavefunction having more amplitude on the gold. The atomic spectrum of neutral and singly ionized Au have many strong or persistent lines. Evidence for these lines can be noted in the featured and asymmetric excitation spectrum obtained from samples prepared using MOA (FIG. 13A). The fact that excitation using any of these wavelengths leads only to LMMCT emission at 610 nm confirms that the ligand metal complexes are strongly associated with both neutral and Au (I) gold atoms. An electronic energy diagram illustrating this relationship is shown in FIG. 13B. As a result of this electronic interaction, there is also enhanced stabilization of the photoluminescent complex under UV excitation, over what has been reported for other gold-thiol complexes in free solution without the presence of nanoparticles. Absorbance was substantially unchanged over 20 minutes exposure to a 300W xenon arc lamp.

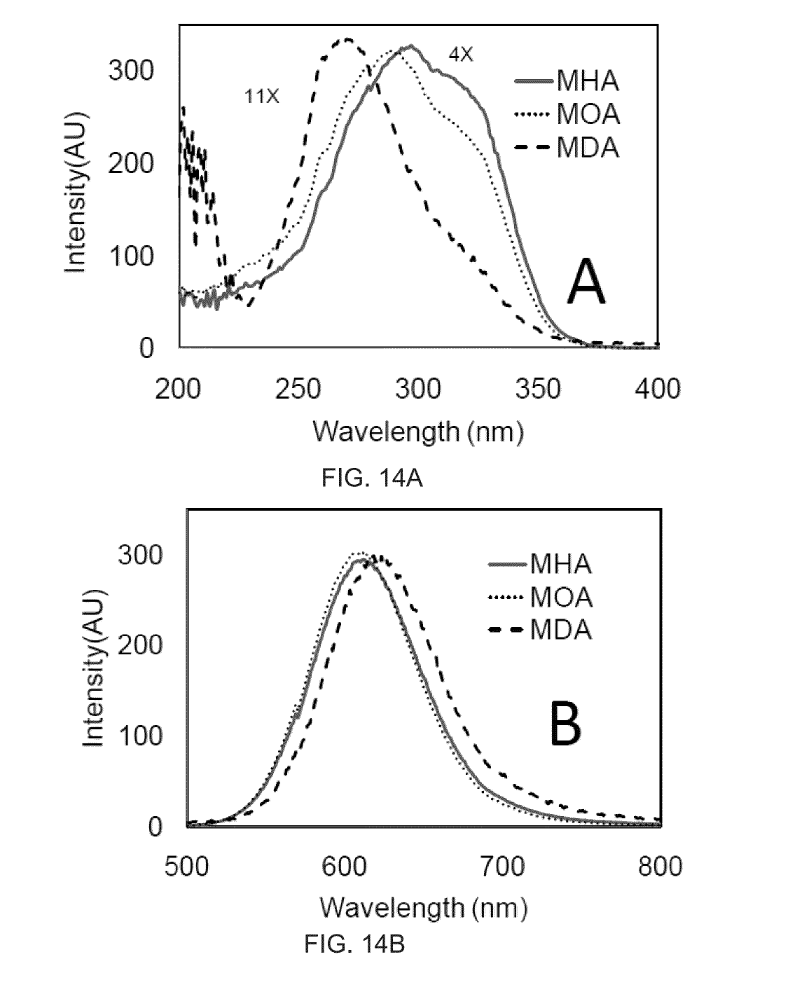

Changing the chain length of the mercaptoalkane carboxylic acid ligand resulted in only a very modest shift in emission wavelength, but did result in both a change of shape and blue-shift of the excitation spectrum maximum to higher energy from ˜4.32 eV to ˜4.97 eV (FIGS. 14A and 14B). Variations in photoluminescence intensity were also noted.

A change in particle size was obtained by increasing mercaptoalkane alcohol ligand chain length as shown in Table 1.

TABLE 1

| | | Estimated | | | TEM | Excitation − |
|---|---|---|---|---|---|---|
| Li- | # | Hydro- | Excit | Emission | size | Emission |
| gands | carbons | phobicity* | (eV) | (eV) | (nm) | shift (eV) |
| MPA | 3 | 0.2 | none | none | large | — |
| MHA | 6 | 1.4 | 4.32 | 2.05 | 3.3 ± 0.9 | 2.27 |
| MOA | 8 | 2.4 | 4.36 | 2.03 | 2.2 ± 0.6 | 2.33 |
| MDA | 12 | 4.5 | 4.97 | 2.00 | 1.7 ± 0.3 | 2.97 |

*The logP was ranked by Smiles-logP calculator provided by Molinspiration software.

Accompanying a change of nearly 1000-fold in intensity was a shift in the emission wavelength to shorter (˜500 nm) wavelength as the nanoparticle size was reduced. These observations appear to be related to size dependent confinement effects.

Changing the mercaptoalkane carboxylic acid ligand chain length had no effect on the emission wavelength but did shift the position of the excitation spectrum maximum to higher energy. This shift is thought to be related to changes in the local hydrophobicity at the surface of the gold nanoparticles where the LMMCT site is likely to be located. A greater stabilization of the absorbing state of the CT complex due to dipole-dipole interactions in low hydrophobicity environment should lead to red shift in the excitation spectrum maximum, while less stabilization and a corresponding blue shift should occur in more hydrophobic environments. This situation can be easily imagined if one considers that the longer chain of MDA interacts with the underlying gold core via stronger dispersion forces, and appears to have a collapsed configuration (FIG. 15A), which protects the underlying LMMCT bonds from interaction with the surrounding water. A schematic of this is illustrated in FIG. 15B, while the FIG. 15A shows what can happen in the case of shorter chain ligands.

The solvent in which gold reduction takes place is one parameter which affets the reaction outcome. For example, in water, NaBH₄ forms several products not all of which retain reducing ability. The first product is BH₄— and is the primary reducing species with an oxidation potential of −1.24 eV. Two other competing reactions are present, one which forms insoluble sodium metaborate, NaBO₂ and another which forms tetrahydroxyborate, NaB(OH)₄, a boron oxoanion. NaBO₂ does not directly participate in Au³⁺ reduction. Given that mixing NaBH₄ with water has the potential to form three products: BH₄—, NaBO₂, and NaB(OH)₄—, any situation which increases the concentrations of the latter two at the expense of the BH₄— concentration at the time of mixing can affect gold reduction and nanoparticle growth. Factors expected to play a role include pH, ethanol content, and amount of NaBH₄ initially used. For instance, the formation of sodium metaborate, NaBO₂ is favored in basic solution. Since the pH of the gold-thiol reaction mixture is naturally alkaline ˜8.5 this is a reaction in our system and is responsible for small amounts of insoluble material formed after mixing. Addition of alcohol to the mixture is known to inhibit the formation of NaBO₂ and instead form alkoxyoxoanions.

As expected in the presence of alcohol no insoluble precipitates are formed at the early stages of the reaction. By blocking this reaction with alcohols, the amount of BH₄— available for reaction with gold can be increased, increasing the efficiency of particle nucleation. In conditions where gold is a limiting reagent, this should result in greater numbers of relatively smaller particles detected by TEM. Consistent with this mechanism, smaller gold particles were observed in the final product when 75 vol % ethanol was used and larger gold particles in the final product with small amounts of ethanol (e.g. 10 vol %). Comparing in greater detail 75%, 25% and 10 vol % EtOH, the average particle sizes were 1.5±0.3 nm, 1.7±0.3 nm, and 2.2±0.6 nm, respectively (n=30). Starting with more NaBH₄ had a similar effect on final product particle size which can be attributed to better nucleation. For example, an initial NaBH₄ concentration of 0.375 mmole produced nanoparticles of smaller dimension, 1.8±0.4 nm (n=50), compared to larger concentrations (0.25 mmol) which produced 2.2±0.6 nm particles (n=50). For very low reductant concentrations, 0.2 mmole and lower, few nanoparticles were produced.

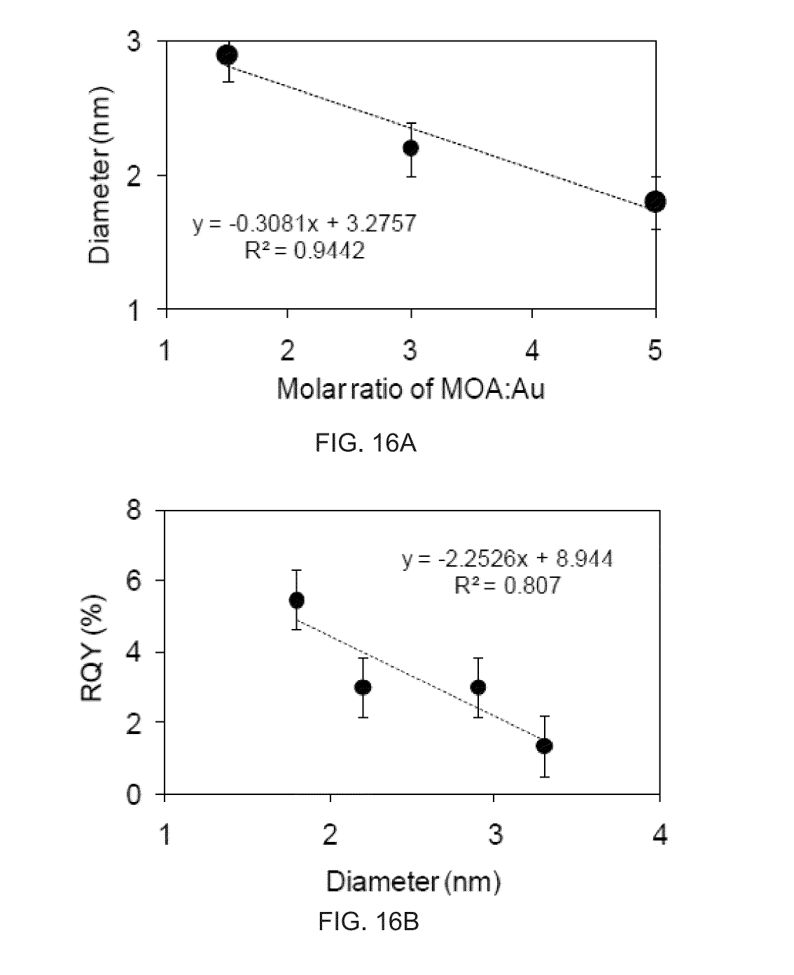

The effect of increasing mercaptoalkane carboxylic acid ligand concentration on particle size was as follows: average particle diameters were 2.9±0.8 nm, 2.2±0.6 nm and 1.8±0.3 nm, for 1.5:1, 3:1, and 5:1 S:Au ratios respectively (n=50)). In reducing conditions, clustered complexes can form what is essentially a nano-sized reactor environment, not unlike that of dendrimers, but with less complex and organized structure. In this environment gold atoms have a chance to react with permeating reductant to nucleate particles. When the initial ligand concentrations are high enough to react with all the gold atoms a maximum number of ligand complexes are produced, and the number of clusters that nucleate will also be high. If reduction is efficient, each of these clusters has the potential to generate a nanoparticle which will be of relatively smaller dimension, since each cluster will have fewer gold atoms in it. On the other hand if the ligand concentration is limiting, then there is likely to be excess gold atoms that can add to any existing cluster increasing their size given sufficient reducing conditions. Consistent with this, the particle diameter increased with decreasing ligand content with an R2 of 0.94 (FIG. 16A). It appears that as the gold atoms reduction takes place there is some rearrangement within a cluster, and the ligand then takes on the role of a capping agent to prevent combination of more than one cluster into larger particles.

FIG. 16B shows that the relative quantum yield of photoluminescence decreases with increasing nanoparticle diameter. According to calculations and experiments, pure gold nanoparticles >2 nm in diameter are not expected to be significantly photoluminescent. However given that the origin of the observed luminescence is from nanoparticles-bound LMMCT complexes, these results can be explained by a reduction in total nanoparticle surface area available for LMMCT complex attachment.

Reactive oxidant species (ROS) formation is an important method to determine biocompatibility since ROS can damage cells and tissues. The ROS formation by gold nanoparticles was tested using Amplex Red assay kit. The result showed that the presence of the gold nanoparticle in Amplex Red solution cause no sign of ROS generation. Interestingly, the result indicates that the gold nanoparticles even showed lowering the ROS comparing to the negative control, water, up to 20% less detection in the case of 0.2 uM gold nanoparticles. This result indicates the possibility of antioxidant activity of the gold nanoparticles. The effect of the metallic oxide nanoparticles on ROS formation and its effect on Amplex Red assay was also dealt as a positive control set. The fluorescence intensity at 610 nm excited using 280 nm light was also found to have a reversible, inverse dependence on temperatures between 1° C. to 35° C.

### CONCLUSIONS

In water the reductive reaction of H₂AuCl₄ and mercaptoalkane carboxylic acids synthesis reaction forms nanoparticle-stabilized photoluminescent ligand complexes of LMMCT character and which are stable against UV irradiation with relatively high photoluminescence yields. No free ligand complexes were detected using electrophoresis, indicating the gold-thiol ligand complexes formed are strongly interacting with gold nanoparticles. Isosbestic features in the time dependent spectra taken during the first 48 hrs of reaction showed that an intense emission was produced as the terminal product of a two state reaction involving the continued reduction of gold-thiol ligand complexes into nanoparticles.

Nanoparticle size was adjustable by altering specific reaction parameters: the chain length and amount of ligand, and the amount of NaBH₄, but no substantial effects on the emission wavelength were noted. Nanoparticle size did affect the photoluminescence yield and the energy maximum of the excitation spectrum. This indicates that the terminal state is the same, but the overlap between the metal and the ligand complex is changing with particle size. Changes in chain length of the ligand had dual effects of changing particle size as well as local hydrophobicity of the emitting species. This affected the energy level of the absorbing species, and the electronic overlap between the CT complex and the gold atoms. Changes in MOA ligand concentration used in the synthesis also affected particle size and excitation spectrum energy maximum in a way that suggests particle size alters the binding of the LMMCT complexes to the particles and possibly the structure of the particles themselves. Two nm diameters is considered to be a transition between ordinary nanoparticles and quantum-enhanced particles. The relative purity and robustness of the materials produced indicates practical applications such as two photon imaging or bioelectronics using these particles. One advantage of this material is that it is a uniquely photostable photoluminescent gold-thiol complex mobilized via a gold nanoparticle platform which is amenable for use in aqueous-based imaging and biomedical applications and are suited for use in two photon and UV energy conversion applications.

The foregoing detailed description describes the invention with reference to specific exemplary embodiments. However, it will be appreciated that various modifications and changes can be made without departing from the scope of the present invention as set forth in the appended claims. The detailed description and accompanying drawings are to be regarded as merely illustrative, rather than as restrictive, and all such modifications or changes, if any, are intended to fall within the scope of the present invention as described and set forth herein.

What is claimed is:

1. A method of detecting pH, comprising:
a) exposing a plurality of nanoparticles to a fluid environment, said nanoparticles having an outer surface functionalized by a carboxy functional group, said nanoparticle being non-toxic and reversibly aggregated as a function of pH;
b) subjecting the plurality of nanoparticles to a light source having a wavelength;
c) measuring a fluorescence intensity of the plurality of nanoparticles; and
d) correlating the fluorescence intensity with a pH.

2. The method of claim 1, wherein the nanoparticle comprises a member selected from the group consisting of gold, silver, platinum, noble metal, iridium, CdS, CdSe, ZrO₂, TiO₂, alloys thereof, intermetallics thereof, and combinations thereof.

3. The method of claim 1, wherein the nanoparticle comprises gold.

4. The method of claim 1, wherein the outer surface is substantially covered by the carboxy functional group.

5. The method of claim 1, wherein the nanoparticle has an average particle diameter from about 1 nm to about 10 nm.

6. The method of claim 1, wherein the nanoparticle has a photobleaching resistance of 10-15% in 2 hours of illumination using a 300W xenon arc lamp.

7. The method of claim 1, wherein the nanoparticle is soluble in an aqueous environment.

8. The method of claim 1, wherein the nanoparticle has a molecular brush structure with differing structural configurations under varying pH.

9. The method of claim 1, wherein the nanoparticle further includes a targeting ligand attached to the outer surface or the carboxy functional group.

10. The method of claim 1, wherein the nanoparticle is capable of use in 2-photon imaging applications.

11. The method of claim 1, wherein the carboxy functional group is selected from the group consisting of mercaptooctanoic acid, mercaptohexanoic acid, mercaptodecanoic acid, mercaptopropanoic acid, and combinations thereof.

12. The method of claim 1, wherein the carboxy functional group is mercaptoalkane carboxylic acid having from six to twelve carbon atoms.

13. The method of claim 1, wherein the fluid environment is a physiological environment.

14. The method of claim 1, wherein the fluid environment is an industrial environment.

15. The method of claim 1, wherein plurality of nanoparticles are present in the fluid environment at a concentration from about 100 nM to about 500 nM.

16. The method of claim 1, wherein the fluorescence intensity is measured using a fluorimeter configured to detect changes in fluorescence of the nanoparticle with changes in pH in a fluid environment.

17. The method of claim 1, wherein the correlating is accomplished by correlating the fluorescence intensity with an assigned intensity on a reference curve with assigned intensities that correlate to pH values.
